(19) United States
(12) Patent Application Publication
(10) Pub. No.: US 2013/0165704 A1
(43) Pub. Date: Jun. 27, 2013

(54) PROCESS FOR PRODUCING ETHANOL IN A REACTOR HAVING A CONSTANT TEMPERATURE

(71) Applicants: Radmila Wollrab, Pasadena, TX (US); Zhenhua Zhou, Houston, TX (US); Victor J. Johnston, Houston, TX (US); Heiko Weiner, Pasadena, TX (US)

(72) Inventors: Radmila Wollrab, Pasadena, TX (US); Zhenhua Zhou, Houston, TX (US); Victor J. Johnston, Houston, TX (US); Heiko Weiner, Pasadena, TX (US)

(73) Assignee: CELANESE INTERNATIONAL CORPORATION, Irving, TX (US)

(21) Appl. No.: 13/585,913

(22) Filed: Aug. 15, 2012

Related U.S. Application Data
(60) Provisional application No. 61/578,612, filed on Dec. 21, 2011.

(51) Int. Cl. C07C 29/149

(52) U.S. Cl. 568/885

(57) ABSTRACT

The present invention, in one embodiment, relates to a process for producing ethanol. The process comprises the step of reacting acetic acid and hydrogen in a shell and tube reactor and in the presence of a catalyst under conditions effective to form a crude ethanol product. The crude ethanol product comprises ethanol, acetic acid, ethyl acetate, and water. The process further comprises the step of recovering ethanol from the crude ethanol product. The shell and tube reactor comprises one or more tubes, each containing a heat transfer medium, and a shell comprising the catalyst. Preferably, the shell and tube reactor has an inlet temperature and an outlet temperature and the inlet temperature is substantially similar to or less than the outlet temperature.

### CROSS REFERENCE TO RELATED APPLICATION

This application claims priority to U.S. Prov. App. No. 61/578,612, filed on Dec. 21, 2011, the entire contents and disclosures of which are incorporated herein by reference.

### FIELD OF THE INVENTION

The present invention relates generally to processes for producing ethanol. In particular, the present invention relates to producing ethanol in a reactor that has a constant temperature. In one embodiment, the reactor may be a shell and tube reactor.

### BACKGROUND OF THE INVENTION

Ethanol for industrial use is conventionally produced from organic feed stocks, such as petroleum oil, natural gas, or coal, from feed stock intermediates, such as syngas, or from starchy materials or cellulosic materials, such as corn or sugar cane. Conventional methods for producing ethanol from organic feed stocks, as well as from cellulosic materials, include the acid-catalyzed hydration of ethylene, methanol homologation, direct alcohol synthesis, and Fischer-Tropsch synthesis. Instability in organic feed stock prices contributes to fluctuations in the cost of conventionally produced ethanol, making the need for alternative sources of ethanol production all the greater when feed stock prices rise. Starchy materials, as well as cellulosic materials, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are formed with ethanol or are formed in side reactions. These impurities limit the production and recovery of ethanol from such reaction mixtures. For example, during hydrogenation, esters are produced that together with ethanol and/or water form azeotropes, which are difficult to separate. In addition when conversion is incomplete, unreacted acid remains in the crude ethanol product, which must be removed to recover ethanol.

EP02060553 describes a process for converting hydrocarbons to ethanol involving converting the hydrocarbons to ethanoic acid and hydrogenating the ethanoic acid to ethanol. The stream from the hydrogenation reactor is separated to obtain an ethanol product and a stream of acetic acid and ethyl acetate, which is recycled to the hydrogenation reactor.

U.S. Pat. No. 4,517,391 describes a hydrogenation process in the presence of cobalt catalyst in a tube reactor, preferably a tube bundle reactor, to provide temperature control.

The need remains for improved processes for efficiently producing ethanol, e.g., with high conversions.

### SUMMARY OF THE INVENTION

In one embodiment, the present invention relates to a process for producing ethanol. The process comprises the step of reacting acetic acid and hydrogen in a shell and tube reactor and in the presence of a catalyst under conditions effective to form a crude ethanol product. The crude ethanol product comprises ethanol, acetic acid, ethyl acetate, and water. The shell and tube reactor comprises one or more tubes, each containing a heat transfer medium, and a shell comprising the catalyst. Preferably, the shell and tube reactor has an inlet temperature and an outlet temperature and the inlet temperature is substantially similar to or less than the outlet temperature. The process further comprises the step of recovering ethanol from the crude ethanol product. The process further comprises the step of maintaining a reaction temperature above a maximum acetic acid evolution temperature, as determined by temperature programmed desorption. In one embodiment, the inlet and/or outlet temperatures are above the maximum acetic acid evolution temperature.

In one embodiment, the present invention relates to a process for producing ethanol in which the reactor is operated at a temperature above a maximum acetic acid evolution temperature, which may be determined by temperature programmed desorption. Preferably, the reactants have a residence time in the reactor and the reactants are at or above the maximum acetic acid evolution temperature for a majority of the residence time.

Preferably, the maximum acetic acid evolution temperature ranges from 200° C. to 350° C. Preferably, the reactor is operated at a temperature from 200° C. to 350° C.

### BRIEF DESCRIPTION OF DRAWINGS

The invention is described in detail below with reference to the appended drawings.

FIG. 1 shows a cross-sectional side view of a shell and tube reactor in accordance with an embodiment of the present invention.

FIG. 2 is a schematic diagram of an ethanol production process in accordance with an embodiment of the present invention.

FIG. 3 is a schematic diagram of another ethanol production process in accordance with an embodiment of the present invention.

FIG. 4 is a schematic diagram of another ethanol production process in accordance with an embodiment of the present invention.

FIG. 5 is a graph showing a signal relating to desorption of the gas from a catalyst surface plotted against temperature.

### DETAILED DESCRIPTION OF THE INVENTION

### Introduction

The present invention relates to processes for producing ethanol via the hydrogenation of acetic acid in the presence of a catalyst. The hydrogenation reaction produces a crude ethanol product that comprises ethanol, water, acetic acid, and other impurities such as ethyl acetate, acetaldehyde, and diethyl acetal. The hydrogenation reaction is exothermic and temperatures may vary in the reactor if the temperatures are not controlled. Although acetic acid may still be converted to ethanol in these varying temperatures, acetic acid conversion may be greater when the reactants are exposed to a temperature that is at or above a maximum acetic acid evolution temperature for a majority of the reactor residence time. The maximum acetic acid evolution temperature is determined by temperature programmed desorption. Preferably, the maximum acetic acid evolution temperature is determined for a catalyst that is exposed to reactant, e.g., has a time-on-stream of at least 5 hours, at least 10 hours or at least 50 hours. Without being bound by theory, when at or above the maximum acetic acid evolution temperature, acetic acid desorbs from the catalyst and may available for reaction with hydrogen to form ethanol. A reactor having temperatures that vary within the reactor may create zones or spots within the reactor that are below the maximum acetic acid evolution temperature. As such, the efficiency of the acetic acid to ethanol conversion is decreased.

The maximum acetic acid evolution temperature may vary depending on the type of catalyst used in the reactor. In most embodiments, the maximum acetic acid evolution temperature is greater than 200° C., e.g., greater than 250° C. or greater than 280° C. In terms of ranges the maximum acetic acid evolution temperature may be from 200° C. to 350° C., e.g., from 270° C. to 350° C. Generally, catalysts that can be used at low temperatures may reduce energy requirements and operating efficiencies. Thus, the preferred reactor temperature ranges are from 200° C. to 350° C., e.g., from 270° C. to 350° C., from 270° C. to 325° C. or from 275° C. to 325° C. In one embodiment, the reactor is operated at 280° C.±5° C. The reactor temperature should be below the acetic acid decomposition temperature.

In preferred embodiments, the reactor has a constant temperature across the reactor, e.g., the temperature difference between the inlet temperature and the outlet temperature is less than 10° C., less than 8° C., less than 5° C., or less than 3° C. Although temperatures may vary, in some embodiments, the inlet temperature may be higher than the outlet temperature. The constant temperature of the reactor allows the reactants to be exposed to the temperature for a majority of the reactor residence time. In a commercially scaled reactor, the residence time at the maximum acetic acid evolution temperature may vary from 5 to 60 seconds, e.g., from 5 to 35 seconds or from 5 to 25 seconds.

In one embodiment, a suitable reactor for maintaining a constant temperature may be a shell and tube reactor. A shell and tube reactor has a tube portion comprising one or more tubes and a shell section. Preferably, the catalyst is contained within the shell section and a heat transfer medium, e.g., air, water, and/or steam, is fed to the one or more tubes. In other embodiments, the catalyst may be contained within the one or more tubes and the heat transfer medium may be contained in the shell section.

When a constant temperature is maintained across the reactor, e.g., the shell and tube reactor, the reactants are allowed to spend the majority of the residence time at a preferred temperature, which contributes to higher conversions of acetic acid.

### Hydrogenation of Acetic Acid

The process of the present invention may be used with any hydrogenation process for producing ethanol as long as the reaction parameters discussed above are maintained. The materials, catalysts, reaction conditions, and separation processes that may be used in the hydrogenation of acetic acid are described further below.

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Biomass-derived syngas has a detectable ¹⁴C isotope content as compared to fossil fuels such as coal or natural gas. An equilibrium forms in the Earth's atmosphere between constant new formation and constant degradation, and so the proportion of the ¹⁴C nuclei in the carbon in the atmosphere on Earth is constant over long periods. The same distribution ratio n¹⁴C:n¹²C ratio is established in living organisms as is present in the surrounding atmosphere, which stops at death and ¹⁴C decomposes at a half life of about 6000 years. Methanol, acetic acid and/or ethanol formed from biomass-derived syngas would be expected to have a ¹⁴C content that is substantially similar to living organisms. For example, the ¹²C ratio of the methanol, acetic acid and/or ethanol may be from one half to about 1 of the ¹⁴C:¹²C ratio for living organisms. In other embodiments, the syngas, methanol, acetic acid and/or ethanol described herein are derived wholly from fossil fuels, i.e. carbon sources produced over 60,000 years ago, may have no detectable ¹⁴C content.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

Acetic acid fed to the hydrogenation reactor may also comprise other carboxylic acids and anhydrides, as well as aldehyde and/or ketones, such as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

As discussed above, the processes of the present invention preferably employ a shell and tube reactor. In one embodiment, multiple shell and tube reactors may be used. In one embodiment, the inventive process may utilize one or more shell and tube reactors in combination with other types of reactors, e.g., fixed bed reactors, radial flow reactors, and/or fluidized bed reactors. In many cases, the reaction zone may be housed in a single vessel or in a series of vessels with heat exchangers configured therebetween.

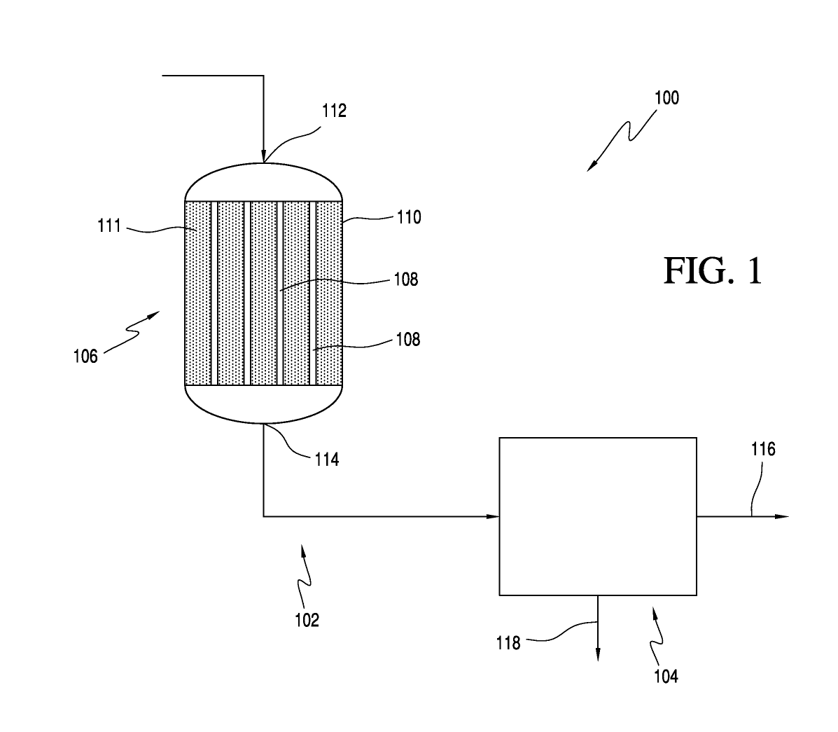

FIG. 1 shows exemplary hydrogenation/separation system 100. The system comprises reaction zone 102 and separation zone 104. The reaction zone comprises shell and tube reactor 106. Reactor 106 comprises one or more tubes 108, which may be collectively referred to as a tube section. Tubes(s) 108 are encompassed by shell section 110, which comprises catalyst 111. Tubes 108 may vary widely in size. In one embodiment, tube(s) have an inner diameter less than 5 cm e.g., less than 2.5 cm, or less than 1 cm. In terms of ranges, the tube inner diameter may range from 0.1 cm to 5 cm, e.g., from 1 cm to 2.5 cm. The outer diameter of the tube(s) may be less than 6 cm, e.g., less than 3.5 cm, or less than 2 cm. Reactor 106 has inlet 112 for receiving reactants and outlet 114 through which the crude ethanol product exits. Although line 112 is shown as being directed to the top of reactor 106, line 112 may be directed to the side, upper portion, or bottom of reactor 106. In one embodiment, the inlet temperature is measured at or around inlet 112 and outlet temperature is measured at or around outlet 114.

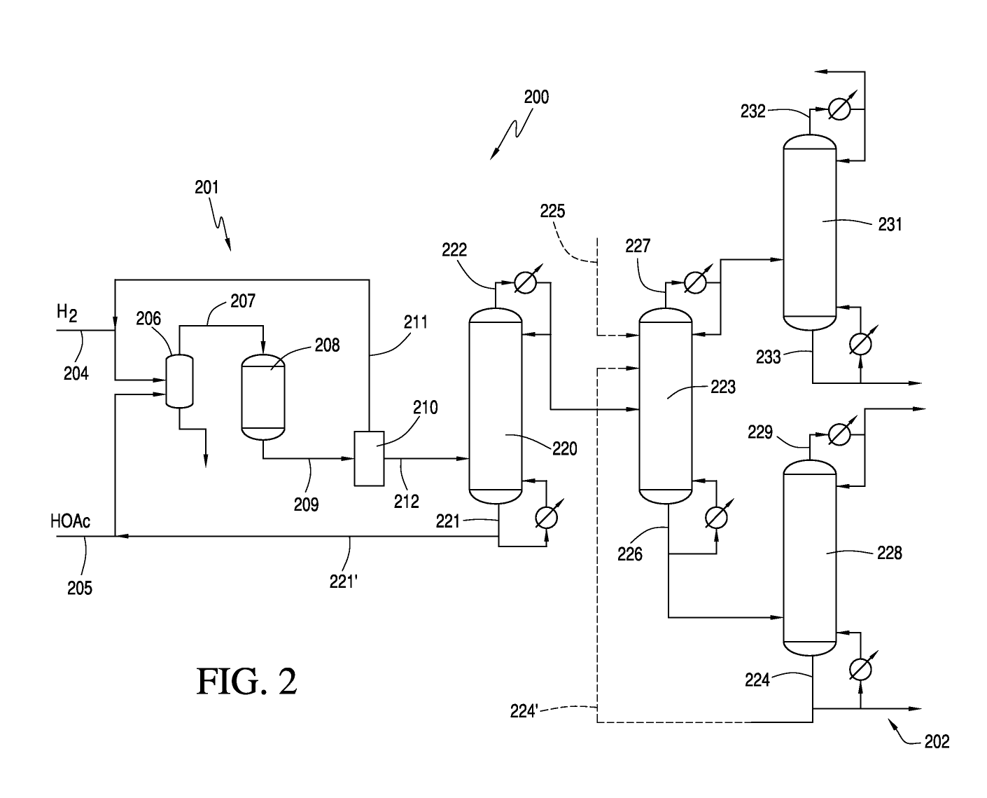

Tubes 108 contain and/or convey a heat transfer medium. The heat transfer medium absorbs and removes heat generated by the reaction. This heat, in some embodiments, may be conveyed via the heat transfer medium to other components of the system, e.g., to the units of separation zone 104 or to the separation columns shown in FIGS. 2-4. The heat transfer medium may vary widely and many heat transfer media are known in the art and are readily available. In one embodiment, the heat transfer medium comprises water, steam, or a combination thereof.

The hydrogenation catalyst is disposed in shell section 110. In operation, the reactants enter the reactor and are conveyed or directed through catalyst bed 111. The reactants react and form a crude ethanol product, which exits via outlet 114. As such, the reactants have a residence time in the reactor. The crude ethanol is directed to separation zone 104, which separated the crude ethanol product into a purified ethanol product, which exits via line 116, and at least one derivative stream, which exits via line 118. Some exemplary separation zones are discussed below.

In one embodiment, the disposition of the heat transfer medium in tube(s) 108 and the catalyst in shell portion 110 beneficially allows for significant reduction in reactor construction materials. As one example, because the heat transfer medium, e.g., pressurized steam, is contained in the tubes (as opposed to the shell), only the tubes require advanced metallurgy. The shell portion typically comprises much more material than the tube portion. As such, when the heat transfer medium is disposed in the tube(s) and not in the shell portion, the shell portion does not require the advanced metallurgy that is conventionally necessary.

In one embodiment, the pressure of heat transfer medium, e.g. steam, within the tube(s) is at least 4000 kPa, e.g., at least 5000 kPa or at least 7000 kPa. In terms of ranges, the pressure in the tube(s) may range from 4000 kPa to 10,000 kPa, e.g., from 6,000 kPa to 9,000 kPa. In one embodiment, the pressure in the shell portion is less than 4000 kPa, e.g., less than 3000 kPa or less than 2500 kPa. In terms of ranges, the pressure in the shell portion may range from 2000 kPa to 4000 kPa, e.g., from 2000 kPa to 3000 kPa.

As discussed above, in some embodiments, the reactor is operated such that, regardless of the operation temperature, there is a constant temperature to which the reactants are exposed, e.g., there is a small (if any) temperature gradient across the reactor. When a constant temperature is maintained across the reactor bed, the reactants are allowed to spend the majority of the residence time at a preferred temperature. In a one embodiment, the preferred temperature is a temperature at or above a maximum acetic acid evolution temperature, which may be determined by temperature programmed desorption (TPD). Accordingly, in one embodiment, the reactor is operated and/or maintained at a temperature at or above the maximum acetic acid evolution temperature. As such, the reactants spend the majority of the residence time at or above the maximum acetic acid evolution temperature, which leads to improved acetic acid conversions.

In one embodiment, within the reactor the reactants are at the preferred temperature, e.g., from 200° C. to 350° C. or at or above the maximum acetic acid evolution temperature, from 5 seconds to 60 seconds, e.g., from 5 seconds to 25 seconds. This residence time in terms of ranges, the reactants are at the preferred temperature for at least 5 seconds, e.g., at least 10 seconds, or at least 15 seconds. In one embodiment, the maximum acetic acid evolution temperature ranges, e.g., from 250° C. to 350°, or from 270° C. to 350° C. Because, in the present invention, the reactants spend significantly more time within the preferred temperature range, acetic acid conversions are markedly improved.

Preferably, the reaction is carried out in the vapor phase under the following conditions. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Exemplary catalysts are further described in U.S. Pat. Nos. 7,608,744 and 7,863,489, and U.S. Pub. Nos. 2010/0121114 and 2010/0197985, the entireties of which are incorporated herein by reference. In another embodiment, the catalyst comprises a Co/Mo/S catalyst of the type described in U.S. Pub. No. 2009/0069609, the entirety of which is incorporated herein by reference. In some embodiments the catalyst may be a bulk catalyst.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In one embodiment, the one or more active metals comprise a first metal selected from the group consisting of copper, iron, cobalt, nickel, ruthenium, rhodium, platinum, palladium, osmium, iridium, titanium, zinc, chromium, rhenium, molybdenum and tungsten. The one or more active metals may further comprise a second metal selected from the group consisting of copper, molybdenum, tin, chromium, iron, cobalt, vanadium, tungsten, palladium, platinum, lanthanum, cerium, manganese, ruthenium, rhenium, gold, and nickel. Preferably, the second metal is different than the first metal.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 7.5 wt. %. For catalysts comprising two or more metals, the two or more metals may be alloyed with one another or may comprise a non-alloyed metal solution or mixture.

Preferred bimetallic metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, cobalt/tin, silver/palladium, copper/palladium, copper/zinc, nickel/palladium, gold/palladium, ruthenium/rhenium, and ruthenium/iron. Additional metal combinations may include palladium/rhenium/tin, palladium/rhenium/cobalt, palladium/rhenium/nickel, platinum/tin/palladium, platinum/tin/cobalt, platinum/tin/copper, platinum/tin/chromium, platinum/tin/zinc, and platinum/tin/nickel.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %. In one embodiment, the catalyst may comprise platinum, tin and cobalt.

In addition to one or more metals, in some embodiments of the present invention the catalysts further comprise a support or a modified support. As used herein, the term “modified support” refers to a support that includes a support material and a support modifier, which adjusts the acidity of the support material.

The total weight of the support or modified support, based on the total weight of the catalyst, preferably is from 75 to 99.9 wt. %, e.g., from 78 to 99 wt. %, or from 80 to 97.5 wt. %. In preferred embodiments that utilize a modified support, the support modifier is present in an amount from 0.1 to 50 wt. %, e.g., from 0.2 to 25 wt. %, from 1 to 20 wt. %, or from 3 to 15 wt. %, based on the total weight of the catalyst. The metals of the catalysts may be dispersed throughout the support, layered throughout the support, coated on the outer surface of the support (i.e., egg shell), or decorated on the surface of the support.

As will be appreciated by those of ordinary skill in the art, support materials are selected such that the catalyst system is suitably active, selective and robust under the process conditions employed for the formation of ethanol.

Suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

In preferred embodiments, the support is selected from the group consisting of silica, silica/alumina, calcium metasilicate, pyrogenic silica, high purity silica, carbon, alumina, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, Co₂O₃, and Bi₂O₃.

In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint-Gobain N or Pro. The Saint-Gobain N or Pro SS61138 silica exhibits the following properties: contains approximately 95 wt. % high surface area silica; surface area of about 250 m²/g; median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0197985 referred to above, the entireties of which are incorporated herein by reference.

In some instances, the hydrogenation catalysts may be used in conjunction with an inert material to regulate the pressure drop of the reactant stream through the catalyst bed and the contact time of the reactant compounds with the catalyst particles.

After the washing, drying and calcining of the catalyst is completed, the catalyst may be reduced in order to activate the catalyst. Reduction is carried out in the presence of a reducing gas, preferably hydrogen. The reducing gas is continuously passed over the catalyst at an initial ambient temperature that is increased up to 400° C. In one embodiment, the reduction is preferably carried out after the catalyst has been loaded into the reaction vessel where the hydrogenation will be carried out.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a percentage based on acetic acid in the feed. The conversion may be at least 40%, e.g., at least 50%, at least 60%, at least 70% or at least 80%. Although catalysts that have high conversions are desirable, such as at least 80% or at least 90%, in some embodiments a low conversion may be acceptable at high selectivity for ethanol.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethanol is at least 60%, e.g., at least 70%, or at least 80%. Preferred embodiments of the hydrogenation process also have low selectivity to undesirable products, such as methane, ethane, and carbon dioxide. The selectivity to these undesirable products preferably is less than 4%, e.g., less than 2% or less than 1%.

The term “productivity,” as used herein, refers to the grams of a specified product, e.g., ethanol, formed during the hydrogenation based on the kilograms of catalyst used per hour. The productivity may range from 100 to 3,000 grams of ethanol per kilogram of catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol stream produced by the reactor, before any subsequent processing, such as purification and separation, will typically comprise unreacted acetic acid, ethanol and water. Exemplary compositional ranges for the crude ethanol product are provided in Table 1, excluding hydrogen. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.2 wt. % to 15 wt. %, from 0.5 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

### Ethanol Separation

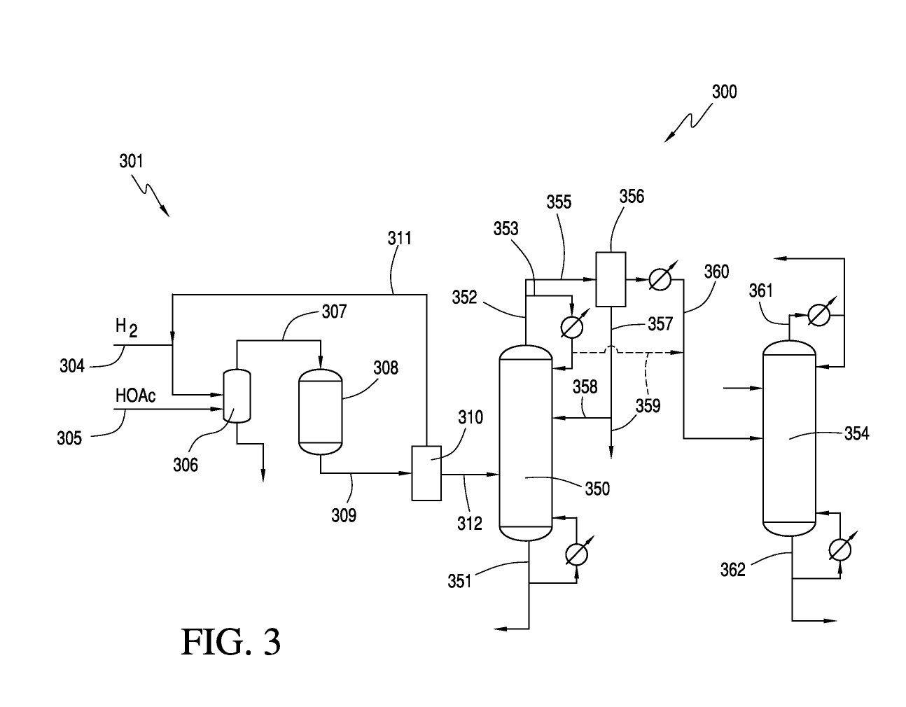

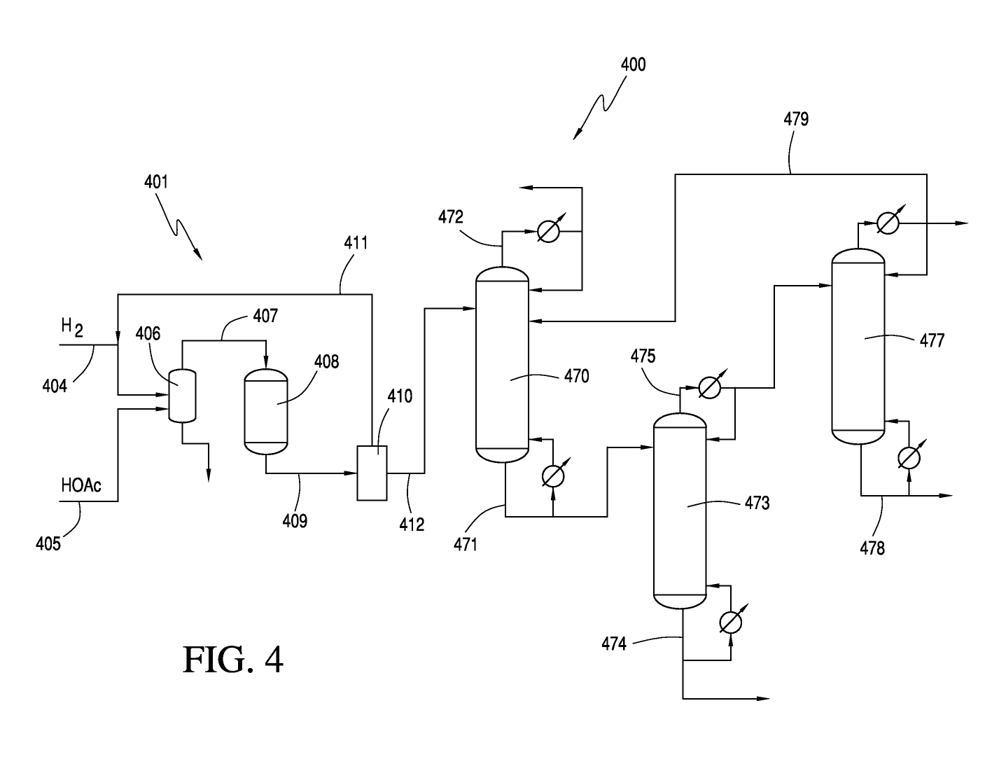

Ethanol produced via the inventive reactor conditions may be recovered using several different techniques. The separation zone of FIG. 2 uses four columns. The separation zone of FIG. 3 employs two columns with an intervening water separation. The separation zone of FIG. 4 uses three columns. Other separation systems may also be used with embodiments of the present invention.

Referring to FIG. 2, hydrogenation system 200 includes a reaction zone 201 and separation zone 202. Hydrogen and acetic acid via lines 204 and 205, respectively, are fed to a vaporizer 206 to create a vapor feed stream in line 207 that is directed to reactor 208. Hydrogen feed line 204 may be preheated to a temperature from 30° C. to 150° C., e.g., from 50° C. to 125° C. or from 60° C. to 115° C. Hydrogen feed line 105 may be fed at a pressure from 1300 kPa to 3100 kPa, e.g., from 1500 kPa to 2800 kPa, or 1700 kPa to 2600 kPa. Acetic acid in line 205 may comprise fresh acetic acid, i.e., acetic acid that has not been previously exposed to a hydrogenation catalyst. Reactor 208 is a shell and tube reactor as discussed above with regard to FIG. 1. In one embodiment, lines 204 and 205 may be combined and jointly fed to vaporizer 206. The temperature of the vapor feed stream in line 207 is preferably from 100° C. to 350° C., e.g., from 120° C. to 310° C. or from 150° C. to 300° C. Any feed that is not vaporized is removed from vaporizer 206 and may be recycled or discarded thereto. In addition, although line 207 is shown as being directed to the top of reactor 208, line 207 may be directed to the side, upper portion, or bottom of reactor 208.

As discussed above, reactor 208 contains the catalyst that is used in the hydrogenation of the carboxylic acid, preferably acetic acid. The catalyst is preferably contained in a shell portion of reactor 208, as noted herein. In one embodiment, one or more guard beds (not shown) may be used upstream of reactor 208, optionally upstream of vaporizer 206, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a crude ethanol product is withdrawn, preferably continuously, from reactor 208 via line 209.

The crude ethanol product in line 209 may be condensed and fed to separator 210, which, in turn, provides a vapor stream 211 and a liquid stream 212. In some embodiments, separator 210 may comprise a flasher or a knockout pot. Separator 210 may operate at a temperature of from 20° C. to 350° C., e.g., from 30° C. to 325° C. or from 60° C. to 250° C. The operating pressure of separator 210 may be from 100 kPa to 3000 kPa, e.g., from 125 kPa to 2500 kPa or from 150 kPa to 2200 kPa. Optionally, the crude ethanol product in line 209 may pass through one or more membranes to separate hydrogen and/or other non-condensable gases.

Vapor stream 211 exiting separator 210 may comprise hydrogen and hydrocarbons, and may be purged and/or returned to reaction zone 201. When returned to reaction zone 201, vapor stream 210 is combined with the hydrogen feed 204 and co-fed to vaporizer 206. In some embodiments, the returned vapor stream 211 may be compressed before being combined with hydrogen feed 204.

In FIG. 2, liquid stream 212 from separator 210 is withdrawn and pumped to the side of first column 220, also referred to as an “acid separation column.” In one embodiment, the contents of liquid stream 212 are substantially similar to the crude ethanol product obtained from the reactor, except that the composition has been depleted of hydrogen, carbon dioxide, methane and/or ethane, which are removed by separator 210. Accordingly, liquid stream 212 may also be referred to as a crude ethanol product. Exemplary components of liquid stream 212 are provided in Table 2. It should be understood that liquid stream 212 may contain other components, not listed in Table 2.

TABLE 2
COLUMN FEED COMPOSITION
(Liquid Stream 112)

| | Conc. | Conc. | Conc. |
|---|---|---|---|
| | (wt. %) | (wt. %) | (wt. %) |
| Ethanol | 5 to 70 | 10 to 60 | 15 to 50 |
| Acetic Acid | <90 | 5 to 80 | 5 to 70 |
| Water | 5 to 30 | 5 to 28 | 10 to 26 |
| Ethyl Acetate | <30 | 0.001 to 20 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout present specification are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 2 may include, but are not limited to, methanol, isopropanol, n-propanol, n-butanol or mixtures thereof. In one embodiment, the liquid stream 212 may comprise propanol, e.g., isopropanol and/or n-propanol, in an amount from 0.001 to 0.1 wt. %, from 0.001 to 0.05 wt. % or from 0.001 to 0.03 wt. %. In should be understood that these other components may be carried through in any of the distillate or residue streams described herein and will not be further described herein, unless indicated otherwise.

Optionally, crude ethanol product in line 209 or in liquid stream 212 may be further fed to an esterification reactor, hydrogenolysis reactor, or combination thereof. An esterification reactor may be used to consume residual acetic acid present in the crude ethanol product to further reduce the amount of acetic acid that would otherwise need to be removed. Hydrogenolysis may be used to convert ethyl acetate in the crude ethanol product to ethanol.

In the embodiment shown in FIG. 2, line 212 is introduced to the lower part of first column 220, e.g., lower half or lower third. In first column 220, unreacted acetic acid, a portion of the water, and other heavy components, if present, are removed from the composition in line 212 and are withdrawn, preferably continuously, as residue in line 221. Some or all of the residue may be returned and/or recycled back to reaction zone 201 via line 221′. Recycling the acetic acid in line 221′ to the vaporizer 206 may reduce the amount of heavies that need to be purged from vaporizer 206. Reducing the amount of heavies to be purged may improve efficiencies of the process while reducing byproducts.

First column 220 also forms an overhead distillate, which is withdrawn in line 222, and which may be condensed and refluxed, for example, at a ratio of from 10:1 to 1:10, e.g., from 3:1 to 1:3 or from 1:2 to 2:1.

When column 220 is operated under standard atmospheric pressure, the temperature of the residue exiting in line 221 preferably is from 95° C. to 120° C., e.g., from 110° C. to 117° C. or from 111° C. to 115° C. The temperature of the distillate exiting in line 222 preferably is from 70° C. to 110° C., e.g., from 75° C. to 95° C. or from 80° C. to 90° C. First column 220 preferably operates at ambient pressure. In other embodiments, the pressure of first column 220 may range from 0.1 kPa to 510 kPa, e.g., from 1 kPa to 475 kPa or from 1 kPa to 375 kPa. Exemplary components of the distillate and residue compositions for first column 220 are provided in Table 3 below. It should also be understood that the distillate and residue may also contain other components, not listed, such as components in the feed. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 3
ACID COLUMN 120 (FIG. 1)

| | Conc. | Conc. | Conc. |
|---|---|---|---|
| | (wt. %) | (wt. %) | (wt. %) |
| Distillate | | | |
| Ethanol | 20 to 75 | 30 to 70 | 40 to 65 |
| Water | 10 to 40 | 15 to 35 | 20 to 35 |
| Acetic Acid | <2 | 0.001 to 0.5 | 0.01 to 0.2 |
| Ethyl Acetate | <60 | 5.0 to 40 | 10 to 30 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | 60 to 100 | 70 to 95 | 85 to 92 |
| Water | <30 | 1 to 20 | 1 to 15 |
| Ethanol | <1 | <0.9 | <0.07 |

As shown in Table 3, without being bound by theory, it has surprisingly and unexpectedly been discovered that when any amount of acetal is detected in the feed that is introduced to the acid separation column 220, the acetal appears to decompose in the column such that less or even no detectable amounts are present in the distillate and/or residue.

The distillate in line 222 preferably comprises ethanol, ethyl acetate, and water, along with other impurities, which may be difficult to separate due to the formation of binary and tertiary azeotropes. To further separate distillate, line 222 is introduced to the second column 223, also referred to as the “light ends column,” preferably in the middle part of column 223, e.g., middle half or middle third. Preferably second column 223 is an extractive distillation column, and an extraction agent is added thereto, optionally via line 224, which is a residue another separation unit, e.g., third column. Extractive distillation is a method of separating close boiling components, such as azeotropes, by distilling the feed in the presence of an extraction agent. The extraction agent preferably has a boiling point that is higher than the compounds being separated in the feed. In preferred embodiments, the extraction agent is comprised primarily of water. As indicated above, the first distillate in line 222 that is fed to second column 223 comprises ethyl acetate, ethanol, and water. These compounds tend to form binary and ternary azeotropes, which decrease separation efficiency. As shown, in one embodiment, the extraction agent comprises the third residue in line 224. Preferably, the recycled third residue in line 224 is fed to second column 223 at a point higher than the first distillate in line 222. In one embodiment, the recycled third residue in line 224 is fed near the top of second column 223 or fed, for example, above the feed in line 222 and below the reflux line from the condensed overheads. In a tray column, the third residue in line 224 is continuously added near the top of the second column 223 so that an appreciable amount of the third residue is present in the liquid phase on all of the trays below. In another embodiment, the extraction agent is fed from a source outside of process 200 via optional line 225 to second column 223. Preferably this extraction agent comprises water.

The molar ratio of the water in the extraction agent to the ethanol in the feed to the second column is preferably at least 0.5:1, e.g., at least 1:1 or at least 3:1. In terms of ranges, preferred molar ratios may range from 0.5:1 to 8:1, e.g., from 1:1 to 7:1 or from 2:1 to 6.5:1. Higher molar ratios may be used but with diminishing returns in terms of the additional ethyl acetate in the second distillate and decreased ethanol concentrations in the second column distillate.

In one embodiment, an additional extraction agent, such as water from an external source, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane and chlorinated paraffins, may be added to second column 223. Some suitable extraction agents include those described in U.S. Pat. Nos. 4,379,028, 4,569,726, 5,993,610 and 6,375,807, the entire contents and disclosure of which are hereby incorporated by reference. The additional extraction agent may be combined with the recycled third residue in line 224 and co-fed to the second column 223. The additional extraction agent may also be added separately to the second column 223. In one aspect, the extraction agent comprises an extraction agent, e.g., water, derived from an external source via line 225 and none of the extraction agent is derived from the third residue.

Second column 223 may be a tray or packed column. In one embodiment, second column 223 is a tray column having from 5 to 70 trays, e.g., from 15 to 50 trays or from 20 to 45 trays. Although the temperature and pressure of second column 223 may vary, when at atmospheric pressure the temperature of the second residue exiting in line 226 preferably is from 60° C. to 90° C., e.g., from 70° C. to 90° C. or from 80° C. to 90° C. The temperature of the second distillate exiting in line 227 from second column 223 preferably is from 50° C. to 90° C., e.g., from 60° C. to 80° C. or from 60° C. to 70° C. Column 223 may operate at atmospheric pressure. In other embodiments, the pressure of second column 223 may range from 0.1 kPa to 510 kPa, e.g., from 1 kPa to 475 kPa or from 1 kPa to 375 kPa. Exemplary components for the distillate and residue compositions for second column 223 are provided in Table 4 below. It should be understood that the distillate and residue may also contain other components, not listed, such as components in the feed.

TABLE 4
SECOND COLUMN 223 (FIG. 2)

| | Conc. | Conc. | Conc. |
|---|---|---|---|
| | (wt. %) | (wt. %) | (wt. %) |
| Distillate | | | |
| Ethyl Acetate | 10 to 99 | 25 to 95 | 50 to 93 |
| Acetaldehyde | <25 | 0.5 to 15 | 1 to 8 |
| Water | <25 | 0.5 to 20 | 4 to 16 |
| Ethanol | <30 | 0.001 to 15 | 0.01 to 5 |
| Acetal | <5 | 0.001 to 2 | 0.01 to 1 |
| Residue | | | |
| Water | 30 to 90 | 40 to 85 | 50 to 85 |
| Ethanol | 10 to 75 | 15 to 60 | 20 to 50 |
| Ethyl Acetate | <3 | 0.001 to 2 | 0.001 to 0.5 |
| Acetic Acid | <0.5 | 0.001 to 0.3 | 0.001 to 0.2 |

In preferred embodiments, the use of an extraction agent, such as the recycling of the third residue, as discussed in detail below, promotes the separation of ethyl acetate from the residue of the second column 223. For example, the weight ratio of ethyl acetate in the second residue to second distillate preferably is less than 0.4:1, e.g., less than 0.2:1 or less than 0.1:1. In embodiments that use an extractive distillation column with water as an extraction agent as the second column 223, the weight ratio of ethyl acetate in the second residue to ethyl acetate in the second distillate approaches zero.

The weight ratio of ethanol in the second residue to second distillate preferably is at least 3:1, e.g., at least 6:1, at least 8:1, at least 10:1 or at least 15:1. All or a portion of the third residue is recycled to the second column. In one embodiment, all of the third residue may be recycled until system 200 reaches a steady state and then a portion of the third residue is recycled with the remaining portion being purged from system 200. The composition of the second residue will tend to have lower amounts of ethanol than when the third residue is not recycled. As the third residue is recycled, the composition of the second residue, as provided in Table 4, comprises less than 30 wt. % of ethanol, e.g., less than 20 wt. % or less than 15 wt. %. The majority of the second residue preferably comprises water. Notwithstanding this effect, the extractive distillation step advantageously also reduces the amount of ethyl acetate that is sent to the third column, which is highly beneficial in ultimately forming a highly pure ethanol product.

As shown, the second residue from second column 223, which comprises ethanol and water, is fed via line 226 to third column 228, also referred to as the “product column.” More preferably, the second residue in line 226 is introduced in the lower part of third column 228, e.g., lower half or lower third. Third column 228 recovers ethanol, which preferably is substantially pure with respect to organic impurities and other than the azeotropic water content, as the distillate in line 229. The distillate of third column 228 preferably is refluxed as shown in FIG. 2, for example, at a reflux ratio of from 1:10 to 10:1, e.g., from 1:3 to 3:1 or from 1:2 to 2:1. The third residue in line 224, which comprises primarily water, preferably is returned via line 224′ to second column 223 as an extraction agent as described above. In one embodiment, a first portion of the third residue in line 224 is recycled to second column 223 and a second portion is purged and removed from the system. In one embodiment, once the process reaches steady state, the second portion of water to be purged is substantially similar to the amount water formed in the hydrogenation of acetic acid. In one embodiment, a portion of the third residue may be used to hydrolyze any other stream, such as one or more streams comprising ethyl acetate.

Although FIG. 2 shows the third residue being directly recycled to second column 223, third residue may also be returned indirectly, for example, by storing a portion or all of the third residue in a tank (not shown) or treating the third residue to further separate any minor components such as aldehydes, higher molecular weight alcohols, or esters in one or more additional columns (not shown).

Third column 228 is preferably a tray column as described above and operates at atmospheric pressure or optionally at pressures above or below atmospheric pressure. The temperature of the third distillate exiting in line 229 preferably is from 60° C. to 110° C., e.g., from 70° C. to 100° C. or from 75° C. to 95° C. The temperature of the third residue in line 224 preferably is from 70° C. to 115° C., e.g., from 80° C. to 110° C. or from 85° C. to 105° C. Exemplary components of the distillate and residue compositions for third column 228 are provided in Table 5 below. It should be understood that the distillate and residue may also contain other components, not listed, such as components in the feed.

TABLE 5
THIRD COLUMN 228 (FIG. 2)

| | Conc. | Conc. | Conc. |
|---|---|---|---|
| | (wt. %) | (wt. %) | (wt. %) |
| Distillate | | | |
| Ethanol | 75 to 96 | 80 to 96 | 85 to 96 |
| Water | <12 | 1 to 9 | 3 to 8 |
| Acetic Acid | <12 | 0.0001 to 0.1 | 0.005 to 0.05 |
| Ethyl Acetate | <12 | 0.0001 to 0.05 | 0.005 to 0.025 |
| Acetaldehyde | <12 | 0.0001 to 0.1 | 0.005 to 0.05 |
| Diethyl Acetal | <12 | 0.0001 to 0.05 | 0.005 to 0.025 |
| Residue | | | |
| Water | 75 to 100 | 80 to 100 | 90 to 100 |
| Ethanol | <0.8 | 0.001 to 0.5 | 0.005 to 0.05 |
| Ethyl Acetate | <1 | 0.001 to 0.5 | 0.005 to 0.2 |
| Acetic Acid | <2 | 0.001 to 0.5 | 0.005 to 0.2 |

In one embodiment, the third residue in line 224 is withdrawn from third column 228 at a temperature higher than the operating temperature of the second column 223. Preferably, the third residue in line 224 is integrated to heat one or more other streams or is reboiled prior to be returned to the second column 223.

Any of the compounds that are carried through the distillation process from the feed or crude reaction product generally remain in the third distillate in amounts of less 0.1 wt. %, based on the total weight of the third distillate composition, e.g., less than 0.05 wt. % or less than 0.02 wt. %. In one embodiment, one or more side streams may remove impurities from any of the columns in system 200. Preferably at least one side stream is used to remove impurities from the third column 228. The impurities may be purged and/or retained within system 200.

The third distillate in line 229 may be further purified to form an anhydrous ethanol product stream, i.e., “finished anhydrous ethanol,” using one or more additional separation systems, such as, for example, distillation columns, adsorption units, membranes, or molecular sieves. Suitable adsorption units include pressure swing adsorption units and thermal swing adsorption unit.

Returning to second column 223, the second distillate preferably is refluxed as shown in FIG. 2, optionally at a reflux ratio of 1:10 to 10:1, e.g., from 1:5 to 5:1 or from 1:3 to 3:1. The second distillate in line 227 may be purged or recycled to the reaction zone. In one embodiment, the second distillate in line 227 is further processed in fourth column 231, also referred to as the “acetaldehyde removal column.” In fourth column 231, the second distillate is separated into a fourth distillate, which comprises acetaldehyde, in line 232 and a fourth residue, which comprises ethyl acetate, in line 233. The fourth distillate preferably is refluxed at a reflux ratio of from 1:20 to 20:1, e.g., from 1:15 to 15:1 or from 1:10 to 10:1, and a portion of the fourth distillate may be returned to the reaction zone 201 (not shown). For example, the fourth distillate may be combined with the acetic acid feed, added to vaporizer 206, or added directly to the reactor 208. The fourth distillate preferably is co-fed with the acetic acid in feed line 205 to vaporizer 206. Without being bound by theory, since acetaldehyde may be hydrogenated to form ethanol, the recycling of a stream that contains acetaldehyde to the reaction zone increases the yield of ethanol and decreases byproduct and waste generation. In another embodiment, the acetaldehyde may be collected and utilized, with or without further purification, to make useful products including but not limited to n-butanol, 1,3-butanediol, and/or crotonaldehyde and derivatives.

The fourth residue of fourth column 231 may be purged via line 233. The fourth residue primarily comprises ethyl acetate and ethanol, which may be suitable for use as a solvent mixture or in the production of esters. In one preferred embodiment, the acetaldehyde is removed from the second distillate in fourth column 231 such that no detectable amount of acetaldehyde is present in the residue of column 231.

Fourth column 231 is preferably a tray column as described above and preferably operates above atmospheric pressure. In one embodiment, the pressure is from 120 kPa to 5,000 kPa, e.g., from 200 kPa to 4,500 kPa, or from 400 kPa to 3,000 kPa. In a preferred embodiment the fourth column 231 may operate at a pressure that is higher than the pressure of the other columns.

The temperature of the fourth distillate exiting in line 232 preferably is from 60° C. to 110° C., e.g., from 70° C. to 100° C. or from 75° C. to 95° C. The temperature of the residue in line 233 preferably is from 70° C. to 115° C., e.g., from 80° C. to 110° C. or from 85° C. to 110° C. Exemplary components of the distillate and residue compositions for fourth column 231 are provided in Table 6 below. It should be understood that the distillate and residue may also contain other components, not listed, such as components in the feed.

TABLE 6
FOURTH COLUMN 231 (FIG. 2)

| | Conc. | Conc. | Conc. |
|---|---|---|---|
| | (wt. %) | (wt. %) | (wt. %) |
| Distillate | | | |
| Acetaldehyde | 2 to 80 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Residue | | | |
| Ethyl Acetate | 40 to 100 | 50 to 100 | 60 to 100 |
| Ethanol | <40 | 0.001 to 30 | 0.01 to 15 |
| Water | <25 | 0.001 to 20 | 2 to 15 |
| Acetaldehyde | <1 | 0.001 to 0.5 | Not detectable |
| Acetal | <3 | 0.001 to 2 | 0.01 to 1 |

In one embodiment, a portion of the third residue in line 224 is recycled to second column 223. In one embodiment, recycling the third residue further reduces the aldehyde components in the second residue and concentrates these aldehyde components in second distillate in line 227 and thereby sent to the fourth column 231, wherein the aldehydes may be more easily separated. The third distillate in line 229 may have lower concentrations of aldehydes and esters due to the recycling of third residue in line 224.

FIG. 3 illustrates another exemplary separation system. The reaction zone 301 of FIG. 3 is similar to that of FIG. 2 and similar numbers indicate similar items. Reaction zone 301 produces liquid stream 312, e.g., crude ethanol product. In one preferred embodiment, reaction zone 301 of FIG. 3 preferably operates at above 80% acetic acid conversion, e.g., above 90% conversion or above 99% conversion. Thus, the acetic acid concentration in the liquid stream 312 may be low.

Liquid stream 312 is introduced in the middle or lower portion of first column 350, also referred to as acid-water column. For purposes of convenience, the columns in each exemplary separation process, may be referred as the first, second, third, etc., columns, but it is understood that first column 350 in FIG. 3 operates differently than the first column 220 of FIG. 2. In one embodiment, no entrainers are added to first column 350. In FIG. 3, first column 350, water and unreacted acetic acid, along with any other heavy components, if present, are removed from liquid stream 312 and are withdrawn, preferably continuously, as a first residue in line 351. Preferably, a substantial portion of the water in the crude ethanol product that is fed to first column 350 may be removed in the first residue, for example, up to about 75% or to about 90% of the water from the crude ethanol product. First column 350 also forms a first distillate, which is withdrawn in line 352.

When column 350 is operated under about 170 kPa, the temperature of the residue exiting in line 351 preferably is from 90° C. to 130° C., e.g., from 95° C. to 120° C. or from 100° C. to 115° C. The temperature of the distillate exiting in line 352 preferably is from 60° C. to 90° C., e.g., from 65° C. to 85° C. or from 70° C. to 80° C. In some embodiments, the pressure of first column 350 may range from 0.1 kPa to 510 kPa, e.g., from 1 kPa to 475 kPa or from 1 kPa to 375 kPa.

The first distillate in line 352 comprises water, in addition to ethanol and other organics. In terms of ranges, the concentration of water in the first distillate in line 352 preferably is from 4 wt. % to 38 wt. %, e.g., from 7 wt. % to 32 wt. %, or from 7 to 25 wt. %. A portion of first distillate in line 353 may be condensed and refluxed, for example, at a ratio of from 10:1 to 1:10, e.g., from 3:1 to 1:3 or from 1:2 to 2:1. It is understood that reflux ratios may vary with the number of stages, feed locations, column efficiency and/or feed composition. Operating with a reflux ratio of greater than 3:1 may be less preferred because more energy may be required to operate the first column 350. The condensed portion of the first distillate may also be fed to second column 354.

The remaining portion of the first distillate in 355 is fed to a water separation unit 356. Water separation unit 356 may be an adsorption unit, membrane, molecular sieves, extractive column distillation, or a combination thereof. A membrane or an array of membranes may also be employed to separate water from the distillate. The membrane or array of membranes may be selected from any suitable membrane that is capable of removing a permeate water stream from a stream that also comprises ethanol and ethyl acetate.

In a preferred embodiment, water separator 356 is a pressure swing adsorption (PSA) unit. The PSA unit is optionally operated at a temperature from 30° C. to 160° C., e.g., from 80° C. to 140° C., and a pressure of from 0.01 kPa to 550 kPa, e.g., from 1 kPa to 150 kPa. The PSA unit may comprise two to five beds. Water separator 356 may remove at least 95% of the water from the portion of first distillate in line 355, and more preferably from 95% to 99.99% of the water from the first distillate, in a water stream 357. All or a portion of water stream 357 may be returned to column 350 in line 358, where the water preferably is ultimately recovered from column 350 in the first residue in line 351. Additionally or alternatively, all or a portion of water stream 357 may be purged via line 359. The remaining portion of first distillate exits the water separator 356 as ethanol mixture stream 360. Ethanol mixture stream 360 may have a low concentration of water of less than 10 wt. %, e.g., less than 6 wt. % or less than 2 wt. %. Exemplary components of ethanol mixture stream 360 and first residue in line 351 are provided in Table 7 below. It should also be understood that these streams may also contain other components, not listed, such as components derived from the feed.

TABLE 7
FIRST COLUMN 350 WITH PSA (FIG. 3)

| | Conc. | Conc. | Conc. |
|---|---|---|---|
| | (wt. %) | (wt. %) | (wt. %) |
| Ethanol Mixture Stream | | | |
| Ethanol | 20 to 95 | 30 to 95 | 40 to 95 |
| Water | <10 | 0.01 to 6 | 0.1 to 2 |
| Acetic Acid | <2 | 0.001 to 0.5 | 0.01 to 0.2 |
| Ethyl Acetate | <60 | 1 to 55 | 5 to 55 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | <90 | 1 to 50 | 2 to 35 |
| Water | 30 to 100 | 45 to 95 | 60 to 90 |
| Ethanol | <1 | <0.9 | <0.3 |

Preferably, ethanol mixture stream 360 is not returned or refluxed to first column 350. The condensed portion of the first distillate in line 353 may be combined with ethanol mixture stream 360 to control the water concentration fed to the second column 354. For example, in some embodiments the first distillate may be split into equal portions, while in other embodiments, all of the first distillate may be condensed or all of the first distillate may be processed in the water separation unit. In FIG. 3, the condensed portion in line 353 and ethanol mixture stream 360 are co-fed to second column 354. In other embodiments, the condensed portion in line 353 and ethanol mixture stream 360 may be separately fed to second column 354. The combined distillate and ethanol mixture has a total water concentration of greater than 0.5 wt. %, e.g., greater than 2 wt. % or greater than 5 wt. %. In terms of ranges, the total water concentration of the combined distillate and ethanol mixture may be from 0.5 to 15 wt. %, e.g., from 2 to 12 wt. %, or from 5 to 10 wt. %.

The second column 354 in FIG. 3, also referred to as the “light ends column,” removes ethyl acetate and acetaldehyde from the first distillate in line 353 and/or ethanol mixture stream 360. Ethyl acetate and acetaldehyde are removed as a second distillate in line 361 and ethanol is removed as the second residue in line 362. Second column 354 may be a tray column or packed column. In one embodiment, second column 354 is a tray column having from 5 to 70 trays, e.g., from 15 to 50 trays or from 20 to 45 trays.

Second column 354 operates at a pressure ranging from 0.1 kPa to 510 kPa, e.g., from 10 kPa to 450 kPa or from 50 kPa to 350 kPa. Although the temperature of second column 354 may vary, when at about 20 kPa to 70 kPa, the temperature of the second residue exiting in line 362 preferably is from 30° C. to 75° C., e.g., from 35° C. to 70° C. or from 40° C. to 65° C. The temperature of the second distillate exiting in line 361 preferably is from 20° C. to 55° C., e.g., from 25° C. to 50° C. or from 30° C. to 45° C.

The total concentration of water fed to second column 354 preferably is less than 10 wt. %, as discussed above. When first distillate in line 353 and/or ethanol mixture stream 360 comprises minor amounts of water, e.g., less than 1 wt. % or less than 0.5 wt. %, additional water may be fed to the second column 354 as an extractive agent in the upper portion of the column. A sufficient amount of water is preferably added via the extractive agent such that the total concentration of water fed to second column 354 is from 1 to 10 wt. % water, e.g., from 2 to 6 wt. %, based on the total weight of all components fed to second column 354. If the extractive agent comprises water, the water may be obtained from an external source or from an internal return/recycle line from one or more of the other columns or water separators.

Suitable extractive agents may also include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof. When extractive agents are used, a suitable recovery system, such as a further distillation column, may be used to recycle the extractive agent.

Exemplary components for the second distillate and second residue compositions for the second column 354 are provided in Table 8, below. It should be understood that the distillate and residue may also contain other components, not listed in Table 8.

TABLE 8
SECOND COLUMN 354 (FIG. 3)

| | Conc. | Conc. | Conc. |
|---|---|---|---|
| | (wt. %) | (wt. %) | (wt. %) |
| Second Distillate | | | |
| Ethyl Acetate | 5 to 90 | 10 to 80 | 15 to 75 |
| Acetaldehyde | <60 | 1 to 40 | 1 to 35 |
| Ethanol | <45 | 0.001 to 40 | 0.01 to 35 |
| Water | <20 | 0.01 to 10 | 0.1 to 5 |
| Second Residue | | | |
| Ethanol | 80 to 99.5 | 85 to 97 | 60 to 95 |
| Water | <20 | 0.001 to 15 | 0.01 to 10 |
| Ethyl Acetate | <1 | 0.001 to 2 | 0.001 to 0.5 |
| Acetic Acid | <0.5 | <0.01 | 0.001 to 0.01 |
| Acetal | <0.05 | <0.03 | <0.01 |

The second residue in FIG. 3 comprises one or more impurities selected from the group consisting of ethyl acetate, acetic acid, acetaldehyde, and diethyl acetal. The second residue may comprise at least 100 wppm of these impurities, e.g., at least 250 wppm or at least 500 wppm. In some embodiments, the second residue may contain substantially no ethyl acetate or acetaldehyde.

The second distillate in line 361, which comprises ethyl acetate and/or acetaldehyde, preferably is refluxed as shown in FIG. 2, for example, at a reflux ratio of from 1:30 to 30:1, e.g., from 1:10 to 10:1 or from 1:3 to 3:1. In one aspect, not shown, the second distillate 361 or a portion thereof may be returned to reactor 308, directly or indirectly via vaporizer 306. The ethyl acetate and/or acetaldehyde in the second distillate may be further reacted in reactor 308.

In one embodiment, the second distillate in line 361 and/or a refined second distillate, or a portion of either or both streams, may be further separated to produce an acetaldehyde-containing stream and an ethyl acetate-containing stream. This may allow a portion of either the resulting acetaldehyde-containing stream or ethyl acetate-containing stream to be recycled to reactor 108 while purging the other stream. The purge stream may be valuable as a source of either ethyl acetate and/or acetaldehyde.

FIG. 4 shows another exemplary separation system. The reaction zone 401 of FIG. 4 is similar to that of FIGS. 2 and 3 and similar numbers indicate similar items. Reaction zone 401 produces liquid stream 412, e.g., crude ethanol product, for further separation. In one preferred embodiment, the reaction zone 401 of FIG. 4 operates at above 80% acetic acid conversion, e.g., above 90% conversion or above 99% conversion. Thus, the acetic acid concentration in the liquid stream 412 may be low.

In the exemplary embodiment shown in FIG. 4, liquid stream 412 is introduced in the upper part of first column 470, e.g., upper half or upper third. In addition to liquid stream 412, an optional extractive agent (not shown) and an optional ethyl acetate recycle stream in line 479 may also be fed to first column 470. The optional extractive agent may comprise water that is introduced above the feed location of the liquid stream 412. In some embodiment, the optional extractive agent may be a dilute acid stream comprising up to 20 wt. % acetic acid. Also, the optional ethyl acetate recycle stream may have a relatively high ethanol concentration, e.g. from 70 to 90 wt. %, and may be fed above or near the feed point of the liquid stream 412.

In one embodiment, first column 470 is a tray column having from 5 to 90 theoretical trays, e.g. from 10 to 60 theoretical trays or from 15 to 50 theoretical trays. The number of actual trays for each column may vary depending on the tray efficiency, which is typically from 0.5 to 0.7 depending on the type of tray. The trays may be sieve trays, fixed valve trays, movable valve trays, or any other suitable design known in the art. In other embodiments, a packed column having structured packing or random packing may be employed.

When first column 470 is operated under 50 kPa, the temperature of the residue exiting in line 471 preferably is from 20° C. to 100° C., e.g., from 30° C. to 90° C. or from 40° C. to 80° C. The base of column 470 may be maintained at a relatively low temperature by withdrawing a residue stream comprising ethanol, ethyl acetate, water, and acetic acid, thereby providing an energy efficiency advantage. The temperature of the distillate exiting in line 472 preferably at 50 kPa is from 10° C. to 80° C., e.g., from 20° C. to 70° C. or from 30° C. to 60° C. The pressure of first column 470 may range from 0.1 kPa to 510 kPa, e.g., from 1 kPa to 475 kPa or from 1 kPa to 375 kPa. In some embodiments, first column 470 may operate under a vacuum of less than 70 kPa, e.g., less than 50 kPa, or less than 20 kPa. Operating under a vacuum may decrease the reboiler duty and reflux ratio of first column 470. However, a decrease in operating pressure for first column 470 does not substantially affect column diameter.

In first column 470, a weight majority of the ethanol, water, acetic acid, are removed from an organic feed, which comprises liquid stream 412 and the optional ethyl acetate recycle stream in line 479, and are withdrawn, preferably continuously, as residue in line 471. This includes any water added as the optional extractive agent. Concentrating the ethanol in the residue reduces the amount of ethanol that is recycled to reactor 408 and in turn reduces the size of reactor 408. Preferably less than 10% of the ethanol from the organic feed, e.g., less than 5% or less than 1% of the ethanol, is returned to reactor 108 from first column 470. In addition, concentrating the ethanol also will concentrate the water and/or acetic acid in the residue. In one embodiment, at least 90% of the ethanol from the organic feed is withdrawn in the residue, and more preferably at least 95%. In addition, ethyl acetate may also be present in the first residue in line 471. The reboiler duty may decrease with an ethyl acetate concentration increase in the first residue in line 471.

First column 470 also forms a distillate, which is withdrawn in line 472, and which may be condensed and refluxed, for example, at a ratio from 30:1 to 1:30, e.g., from 10:1 to 1:10 or from 5:1 to 1:5. Higher mass flow ratios of water to organic feed may allow first column 470 to operate with a reduced reflux ratio.

First distillate in line 472 preferably comprises a weight majority of the acetaldehyde and ethyl acetate from liquid stream 412, as well as from the optional ethyl acetate recycle stream in line 479. In one embodiment, the first distillate in line 472 comprises a concentration of ethyl acetate that is less than the ethyl acetate concentration for the azeotrope of ethyl acetate and water, and more preferably less than 75 wt. %.

In some embodiments, first distillate in stream 472 also comprises ethanol. Returning the first distillate comprising ethanol to the reactor may require an increase in reactor capacity to maintain the same level of ethanol efficiency. In one embodiment, it is preferred to return to the reactor less than 10% of the ethanol from the crude ethanol stream, e.g., less than 5% or less than 1%. In terms of ranges, the amount of returned ethanol is from 0.01 to 10% of the ethanol in the crude ethanol stream, e.g. from 0.1 to 5% or from 0.2 to 1%. In one embodiment, to reduce the amount of ethanol returned, the ethanol may be recovered from the first distillate in line 472 using an optional extractor or extractive distillation column.

Exemplary components of the distillate and residue compositions for first column 470 are provided in Table 9 below. It should also be understood that the distillate and residue may also contain other components, not listed in Table 9. For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

TABLE 9
FIRST COLUMN 470 (FIG. 4)

| | Conc. | Conc. | Conc. |
|---|---|---|---|
| | (wt. %) | (wt. %) | (wt. %) |
| Distillate | | | |
| Ethyl Acetate | 10 to 85 | 15 to 80 | 20 to 75 |
| Acetaldehyde | 0.1 to 70 | 0.2 to 65 | 0.5 to 65 |
| Acetal | <0.1 | <0.1 | <0.05 |
| Acetone | <0.05 | 0.001 to 0.03 | 0.01 to 0.025 |
| Ethanol | 3 to 55 | 4 to 50 | 5 to 45 |
| Water | 0.1 to 20 | 1 to 15 | 2 to 10 |
| Acetic Acid | <2 | <0.1 | <0.05 |
| Residue | | | |
| Acetic Acid | 0.01 to 50 | 0.5 to 40 | 1 to 30 |
| Water | 5 to 40 | 5 to 35 | 10 to 25 |
| Ethanol | 10 to 75 | 15 to 70 | 20 to 65 |
| Ethyl Acetate | 0.005 to 30 | 0.03 to 25 | 0.08 to 1 |

In an embodiment of the present invention, column 470 may be operated at a temperature where most of the water, ethanol, and acetic acid are removed into the residue stream and only a small amount of ethanol and water is collected in the distillate stream due to the formation of binary and tertiary azeotropes. The weight ratio of water in the residue in line 471 to water in the distillate in line 472 may be greater than 1:1, e.g., greater than 2:1. The weight ratio of ethanol in the residue to ethanol in the distillate may be greater than 1:1, e.g., greater than 2:1

The amount of acetic acid in the first residue may vary depending primarily on the conversion in reactor 408. In one embodiment, when the conversion is high, e.g., greater than 90%, the amount of acetic acid in the first residue may be less than 10 wt. %, e.g., less than 5 wt. % or less than 2 wt. %. In other embodiments, when the conversion is lower, e.g., less than 90%, the amount of acetic acid in the first residue may be greater than 10 wt. %.

The distillate preferably is substantially free of acetic acid, e.g., comprising less than 1000 wppm, less than 500 wppm or less than 100 wppm acetic acid. The distillate may be purged from the system or recycled in whole or part to reactor 408. In some embodiments, the distillate may be further separated, e.g., in a distillation column (not shown), into an acetaldehyde stream and an ethyl acetate stream. Either of these streams may be returned to reactor 408 or separated from system 400 as additional product. The ethyl acetate stream may also be hydrolyzed or reduced with hydrogen, via hydrogenolysis, to produce ethanol. When additional ethanol is produced, it is preferred that the additional ethanol is recovered and not directed to reactor 408.

Some species, such as acetals, may decompose in first column 470 such that very low amounts, or even no detectable amounts, of acetals remain in the distillate or residue.

To recover ethanol, first residue in line 471 may be further separated depending on the concentration of acetic acid and/or ethyl acetate. In FIG. 4, residue in line 471 is further separated in a second column 473, also referred to as an “acid column.” Second column 473 yields a second residue in line 474 comprising acetic acid and water, and a second distillate in line 475 comprising ethanol and ethyl acetate. In one embodiment, a weight majority of the water and/or acetic acid fed to second column 473 is removed in the second residue in line 474, e.g., at least 60% of the water and/or acetic acid is removed in the second residue in line 474 or more preferably at least 80% of the water and/or acetic acid. An acid column may be desirable, for example, when the acetic acid concentration in the first residue is greater 50 wppm, e.g., greater than 0.1 wt. %, greater than 1 wt. %, e.g., greater than 5 wt. %.

In one embodiment, a portion of the first residue in line 471 may be preheated prior to being introduced into second column 473, as shown in FIG. 4. After preheating, first residue in line 471 may be converted into a partial vapor feed having less than 30 mol. % of the contents in the vapor phase, e.g., less than 25 mol. % or less than 20 mol. %. In terms of ranges, from 1 to 30 mol. % is in the vapor phase, e.g., from 5 to 20 mol. %. Greater vapor phase contents result in increased energy consumption and a significant increase in the size of second column 473.

Second column 473 operates in a manner to concentrate the ethanol from first residue so that a majority of the ethanol is carried overhead. Thus, the residue of second column 473 may have a low ethanol concentration of less than 5 wt. %, e.g. less than 1 wt. % or less than 0.5 wt. %. Lower ethanol concentrations may be achieved without significant increases in reboiler duty or column size. Thus, in some embodiments, it is efficient to reduce the ethanol concentration in the residue to less than 50 wppm, or more preferably less than 25 wppm. As described herein, the residue of second column 473 may be treated and lower concentrations of ethanol allow the residue to be treated without generating further impurities.

In FIG. 4, the first residue in line 471 is introduced to second column 473 preferably in the top part of column 473, e.g., top half or top third. Feeding first residue in line 471 in a lower portion of second column 473 may unnecessarily increase the energy requirements. Acid column 473 may be a tray column or packed column. In FIG. 4, second column 473 may be a tray column having from 10 to 110 theoretical trays, e.g. from 15 to 95 theoretical trays or from 20 to 75 theoretical trays. Additional trays may be used if necessary to further reduce the ethanol concentration in the residue. In one embodiment, the reboiler duty and column size may be reduced by increasing the number of trays.

Although the temperature and pressure of second column 473 may vary, when at atmospheric pressure the temperature of the second residue in line 474 preferably is from 95° C. to 160° C., e.g., from 100° C. to 150° C. or from 110° C. to 145° C. In one embodiment, first residue in line 471 is preheated to a temperature that is within 20° C. of the temperature of second residue in line 474, e.g., within 15° C. or within 10° C. The temperature of the second distillate exiting in line 475 from second column 473 preferably is from 50° C. to 120° C., e.g., from 75° C. to 118° C. or from 80° C. to 115° C. The temperature gradient may be sharper in the base of second column 473.

The pressure of second column 473 may range from 0.1 kPa to 510 kPa, e.g., from 1 kPa to 475 kPa or from 1 kPa to 375 kPa. In one embodiment, second column 473 operates above atmospheric pressure, e.g., above 170 kPa or above 375 kPa. Second column 473 may be constructed of a material such as 316L SS, Allot 2205 or Hastelloy C, depending on the operating pressure. The reboiler duty and column size for second column 473 remain relatively constant until the ethanol concentration in the second distillate in line 475 is greater than 90 wt. %.

Second column 473 also forms an overhead, which is withdrawn, and which may be condensed and refluxed, for example, at a ratio from 12:1 to 1:12, e.g., from 10:1 to 1:10 or from 8:1 to 1:8. The overhead preferably comprises 85 to 92 wt. % ethanol, e.g., about 87 to 90 wt. % ethanol, with the remaining balance being water and ethyl acetate. In one embodiment, water may be removed prior to recovering the ethanol product as described above in FIG. 4. In one embodiment, the overhead, prior to water removal, may comprise less than 15 wt. % water, e.g., less than 10 wt. % water or less than 8 wt. % water. Overhead vapor may be fed to water separator, which may be an adsorption unit, membrane, molecular sieves, extractive column distillation, or a combination thereof.

Exemplary components for the distillate and residue compositions for second column 473 are provided in Table 10 below. It should be understood that the distillate and residue may also contain other components, not listed in Table 10. For example, in optional embodiments, when ethyl acetate is in the feed to reactor 408, second residue in line 474 exemplified in Table 10 may also comprise high boiling point components.

TABLE 10
SECOND COLUMN 473 (FIG. 4)

| | Conc. | Conc. | Conc. |
|---|---|---|---|
| | (wt. %) | (wt. %) | (wt. %) |
| Second Distillate | | | |
| Ethanol | 80 to 96 | 85 to 92 | 87 to 90 |
| Ethyl Acetate | <30 | 0.001 to 15 | 0.005 to 4 |
| Acetaldehyde | <20 | 0.001 to 15 | 0.005 to 4 |
| Water | <20 | 0.001 to 10 | 0.01 to 8 |
| Acetal | <2 | 0.001 to 1 | 0.005 to 0.5 |
| Second Residue | | | |
| Acetic Acid | 0.1 to 55 | 0.2 to 40 | 0.5 to 35 |
| Water | 45 to 99.9 | 55 to 99.8 | 65 to 99.5 |
| Ethyl Acetate | <0.1 | 0.0001 to 0.05 | 0.0001 to 0.01 |
| Ethanol | <5 | 0.002 to 1 | 0.005 to 0.5 |

The weight ratio of ethanol in second distillate in line 475 to ethanol in the second residue in line 474 preferably is at least 35:1. Preferably, second distillate in line 475 is substantially free of acetic acid and may contain, if any, trace amounts of acetic acid.

In one embodiment, ethyl acetate fed to second column 473 may concentrate in the second distillate in line 475. Thus, preferably no ethyl acetate is withdrawn in the second residue in line 474. Advantageously this allows most of the ethyl acetate to be subsequently recovered without having to further process the second residue in line 474.

In one embodiment, as shown in FIG. 4, due to the presence of ethyl acetate in second distillate in line 475, an additional third column 477 may be used. Third column 477, referred to as a “product” column, is used for removing ethyl acetate from second distillate in line 475 and producing an ethanol product in the third residue in line 478. Product column 477 may be a tray column or packed column. In FIG. 4, third column 477 may be a tray column having from 5 to 90 theoretical trays, e.g. from 10 to 60 theoretical trays or from 15 to 50 theoretical trays.

The feed location of second distillate in line 475 may vary depending on ethyl acetate concentration and it is preferred to feed second distillate in line 475 to the upper portion of third column 477. Higher concentrations of ethyl acetate may be fed at a higher location in third column 477. The feed location should avoid the very top trays, near the reflux, to avoid excess reboiler duty requirements for the column and an increase in column size. For example, in a column having 45 actual trays, the feed location should between 10 to 15 trays from the top. Feeding at a point above this may increase the reboiler duty and size of third column 477.

Second distillate in line 475 may be fed to third column 477 at a temperature of up to 70° C., e.g., up to 50° C., or up to 40° C. In some embodiments it is not necessary to further preheat second distillate in line 475.

Ethyl acetate may be concentrated in the third distillate in line 479. Due to the relatively lower amounts of ethyl acetate fed to third column 477, third distillate in line 479 also comprises substantial amounts of ethanol. To recover the ethanol, third distillate in line 479 may be fed to first column 470 as an optional ethyl acetate recycle stream 479. Depending on the ethyl acetate concentration of optional ethyl acetate recycle stream 479 this stream may be introduced above or near the feed point of the liquid stream 412. Depending on the targeted ethyl acetate concentration in the distillate of first column 472 the feed point of optional ethyl acetate recycle stream 479 will vary. Liquid stream 412 and optional ethyl acetate recycle stream 479 collectively comprise the organic feed to first column 470. In one embodiment, organic feed comprises from 1 to 25% of optional ethyl acetate recycle stream 179, e.g., from 3% to 20% or from 5% to 15%. This amount may vary depending on the production of reactor 408 and amount of ethyl acetate to be recycled.

Because ethyl acetate recycle stream 479 increases the demands on the first and second columns, it is preferred that the ethanol concentration in third distillate in line 479 be from 70 to 90 wt. %, e.g., from 72 to 88 wt. %, or from 75 to 85 wt. %. In other embodiments, a portion of third distillate in line 479 may be purged from the system as additional products, such as an ethyl acetate solvent. In addition, ethanol may be recovered from a portion of the third distillate in line 479 using an extractant, such as benzene, propylene glycol, and cyclohexane, so that the raffinate comprises less ethanol to recycle.

The third residue in line 479 from third column 477 may comprise ethanol and optionally any remaining water. In an optional embodiment, the third residue may be further processed to recover ethanol with a desired amount of water, for example, using a further distillation column, adsorption unit, membrane or combination thereof, may be used to further remove water from third residue in line 478, as necessary.

Third column 477 is preferably a tray column as described above and preferably operates at atmospheric pressure. The temperature of the third residue exiting from third column 477 preferably is from 65° C. to 110° C., e.g., from 70° C. to 100° C. or from 75° C. to 80° C. The temperature of the third distillate exiting from third column 477 preferably is from 30° C. to 70° C., e.g., from 40° C. to 65° C. or from 50° C. to 65° C.

The pressure of third column 477 may range from 0.1 kPa to 510 kPa, e.g., from 1 kPa to 475 kPa or from 1 kPa to 375 kPa. In some embodiments, third column 477 may operate under a vacuum of less than 70 kPa, e.g., less than 50 kPa, or less than 20 kPa. Decreases in operating pressure substantially decreases column diameter and reboiler duty for third column 176.

Exemplary components for ethanol mixture stream and residue compositions for third column 477 are provided in Table 11 below. It should be understood that the distillate and residue may also contain other components, not listed in Table 11.

TABLE 11
PRODUCT COLUMN (FIG. 3)

| | Conc. | Conc. | Conc. |
|---|---|---|---|
| | (wt. %) | (wt. %) | (wt. %) |
| Third Distillate | | | |
| Ethanol | 70 to 99 | 72 to 95 | 75 to 90 |
| Ethyl Acetate | 1 to 30 | 1 to 25 | 1 to 15 |
| Acetaldehyde | <15 | 0.001 to 10 | 0.1 to 5 |
| Water | <10 | 0.001 to 2 | 0.01 to 1 |
| Acetal | <2 | 0.001 to 1 | 0.01 to 0.5 |
| Third Residue | | | |
| Ethanol | 80 to 99.5 | 85 to 97 | 90 to 95 |
| Water | <3 | 0.001 to 2 | 0.01 to 1 |
| Ethyl Acetate | <1.5 | 0.0001 to 1 | 0.001 to 0.5 |
| Acetic Acid | <0.5 | <0.01 | 0.0001 to 0.01 |

Some of the residues withdrawn from the separation zone(s) comprise acetic acid and water. Depending on the amount of water and acetic acid contained in the residue of first column, e.g., 220 in FIG. 2, 350 in FIG. 3, or residue of second column 473 in FIG. 4, the residue may be treated in one or more of the following processes. The following are exemplary processes for further treating the residue and it should be understood that any of the following may be used regardless of acetic acid concentration. When the residue comprises a majority of acetic acid, e.g., greater than 70 wt. %, the residue may be recycled to the reactor without any separation of the water. In one embodiment, the residue may be separated into an acetic acid stream and a water stream when the residue comprises a majority of acetic acid, e.g., greater than 50 wt. %. Acetic acid may also be recovered in some embodiments from the residue having a lower acetic acid concentration. The residue may be separated into the acetic acid and water streams by a distillation column or one or more membranes. If a membrane or an array of membranes is employed to separate the acetic acid from the water, the membrane or array of membranes may be selected from any suitable acid resistant membrane that is capable of removing a permeate water stream. The resulting acetic acid stream optionally is returned to the reactor 108. The resulting water stream may be used as an extractive agent or to hydrolyze an ester-containing stream in a hydrolysis unit.

In other embodiments, for example, where the residue comprises less than 50 wt. % acetic acid, possible options include one or more of: (i) returning a portion of the residue to reactor 108, (ii) neutralizing the acetic acid, (iii) reacting the acetic acid with an alcohol, or (iv) disposing of the residue in a waste water treatment facility. It also may be possible to separate a residue comprising less than 50 wt. % acetic acid using a weak acid recovery distillation column to which a solvent (optionally acting as an azeotroping agent) may be added. Exemplary solvents that may be suitable for this purpose include ethyl acetate, propyl acetate, isopropyl acetate, butyl acetate, vinyl acetate, diisopropyl ether, carbon disulfide, tetrahydrofuran, isopropanol, ethanol, and C₃-C₁₂ alkanes. When neutralizing the acetic acid, it is preferred that the residue comprises less than 10 wt. % acetic acid. Acetic acid may be neutralized with any suitable alkali or alkaline earth metal base, such as sodium hydroxide or potassium hydroxide. When reacting acetic acid with an alcohol, it is preferred that the residue comprises less than 50 wt. % acetic acid. The alcohol may be any suitable alcohol, such as methanol, ethanol, propanol, butanol, or mixtures thereof. The reaction forms an ester that may be integrated with other systems, such as carbonylation production or an ester production process. Preferably, the alcohol comprises ethanol and the resulting ester comprises ethyl acetate. Optionally, the resulting ester may be fed to the hydrogenation reactor.

In some embodiments, when the residue comprises very minor amounts of acetic acid, e.g., less than 5 wt. %, the residue may be disposed of to a waste water treatment facility without further processing. The organic content, e.g., acetic acid content, of the residue beneficially may be suitable to feed microorganisms used in a waste water treatment facility.

The columns shown in figures may comprise any distillation column capable of performing the desired separation and/or purification. Each column preferably comprises a tray column having from 1 to 150 trays, e.g., from 10 to 100 trays, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. As a practical matter, pressures from 10 kPa to 3000 kPa will generally be employed in these zones although in some embodiments subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The ethanol product produced by the process of the present invention may be an industrial grade ethanol comprising from 75 to 96 wt. % ethanol, e.g., from 80 to 96 wt. % or from 85 to 96 wt. % ethanol, based on the total weight of the ethanol product. Exemplary finished ethanol compositional ranges are provided below in Table 12.

TABLE 12
FINISHED ETHANOL COMPOSITIONS

| | Conc. | Conc. | Conc. |
|---|---|---|---|
| Component | (wt. %) | (wt. %) | (wt. %) |
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit as discussed above. In such embodiments, the ethanol concentration of the ethanol product may be higher than indicated in Table 11, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entireties of which is incorporated herein by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Yin U.S. Pat. No. 3,130,007, the entireties of which are hereby incorporated herein by reference.

### EXAMPLES

### Temperature Programmed Desorption

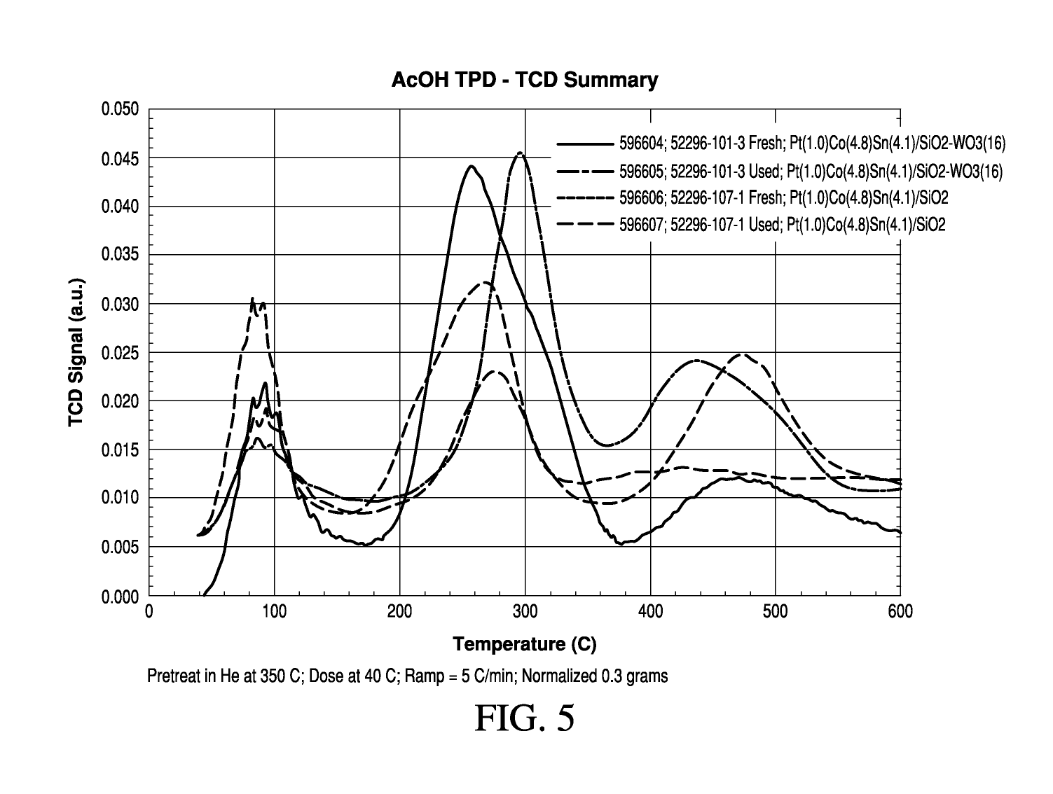

Surface reactions and molecular adsorption on surfaces can be studied using TPD. The TPD technique involves the adsorption of a species on the surface of the catalyst at low temperature, e.g., close to room temperature, and heating the sample at a linear ramp rate while monitoring the species that evolve from the surface of the catalyst. Desorption of the gas from the surface produces a signal in the detector. This signal is plotted against temperature to obtain the TPD plot as shown in FIG. 5. Generally speaking, the area under the peak of the desorbed signal will be proportional to the amount of adsorbed gas. In other words, the area under the curve may be indicative of the surface coverage. The position of peak temperature or onset temperature may be indicative of the strength of adsorption. If there are multiple binding sites on the surface, multiple peak temperatures are observed in the TPD graph.

In some embodiments, TPD experiments may be carried out on catalysts suitable for use with the present invention using acetic acid. For example, 20% acetic acid vapor in helium gas flow at 50 sccm may be pulse dosed on about 0.3 grams of conditioned catalyst held at 40° C. until a saturation amount of adsorption of acetic acid is achieved. Catalyst conditioning may be achieved by heating the catalyst, e.g., to 350° C. for 2 hours, to remove moisture and any surface contaminants. The catalyst may then be cooled, e.g., to 40° C., and pulse adsorption of acetic acid is done. Helium gas at 50 sccm may then be passed over the catalyst to remove any loosely held acetic acid. Catalyst may then be heated at a linear rate, e.g., 5° C./min from 40 to 600° C., and held at that temperature, e.g., for 1 hour. Desorption of acetic acid was monitored using thermal conductivity detector (TCD).

FIG. 5 is an exemplary graph used to determine maximum acetic acid evolution temperature determination. FIG. 5 shows curves of two catalysts that are fresh (596604 and 596606) and curves relating to the same catalysts that have been used (596605 and 596607). Catalysts 596604 and 596605 are Pt(1 wt. %)—Co (4.8 wt. %)—Sn(4.1 wt. %) on silica modified with WO₃(16 wt. %). Catalysts 596606 and 596607 are Pt(1 wt. %)—Co (4.8 wt. %)—Sn(4.1 wt. %) on silica that is not modified. FIG. 5 shows three distinct peaks of temperature that are observed for the exemplary catalysts. The temperature peak at less than 100° C. is typically due to physisorption (weakly bound) and can be ignored. For the 596607 catalyst, a peak temperature is observed at 280° C. Generally, it is preferred to determine the peak temperature based on the used catalyst and not the fresh catalyst. The peak temperature implies that if the reactor is maintained at a temperature at or slightly higher than the peak temperature, most of the acetic acid reactant will be in the vapor phase over the catalyst surface and will react to form the product ethanol. Maximum conversion of acetic acid would be possible if the reaction temperature is held slightly above the peak maximum temperature.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising:
(a) reacting acetic acid and hydrogen in a shell and tube reactor and in the presence of a catalyst under conditions effective to form a crude ethanol product comprising ethanol, acetic acid, ethyl acetate, and water; and
(b) recovering ethanol from the crude ethanol product;
wherein the shell and tube reactor comprises one or more tubes each containing a heat transfer medium and a shell comprising the catalyst; and
wherein the shell and tube reactor has an inlet temperature and an outlet temperature and the inlet temperature is substantially similar to or less than the outlet temperature.

2. The process of claim 1, wherein a temperature difference between the inlet temperature and the outlet temperature is less than 10° C.

3. The process of claim 1, wherein the reactor is operated at a temperature from 200° C. to 350° C.

4. The process of claim 1, further comprising the step of:
maintaining a reaction temperature above a maximum acetic acid evolution temperature, as determined by temperature programmed desorption.

5. The process of claim 4, wherein the maximum acetic acid evolution temperature ranges from 200° C. to 350° C.

6. The process of claim 4, wherein the maximum acetic acid evolution temperature is greater than 280° C.

7. The process of claim 1, wherein overall conversion of acetic acid is at least 75%, based on the total acetic acid fed to the reactor.

8. The process of claim 1, wherein selectivity to ethanol is at least 60%.

9. The process of claim 1, wherein the heat transfer medium comprises water, steam, or a combination thereof.

10. The process of claim 1, wherein the one or more tubes have an inner diameter of less than 5 cm.

11. The process of claim 1, wherein the catalyst comprises one or more active metals on a support.

12. The process of claim 11, wherein the one or more active metals comprise a first metal selected from the group consisting of copper, iron, cobalt, nickel, ruthenium, rhodium, platinum, palladium, osmium, iridium, titanium, zinc, chromium, rhenium, molybdenum and tungsten, and a second metal selected from the group consisting of copper, molybdenum, tin, chromium, iron, cobalt, vanadium, tungsten, palladium, platinum, lanthanum, cerium, manganese, ruthenium, rhenium, gold, and nickel; and further wherein the second metal is different than the first metal.

13. The process of claim 11, wherein the support is selected from the group consisting of silica, silica/alumina, calcium metasilicate, pyrogenic silica, high purity silica, carbon, alumina, and mixtures thereof.

14. The process of claim 1, wherein the catalyst further comprises a support modifier.

15. The catalyst of claim 14, wherein the support modifier is selected from the group consisting of (i) alkaline earth metal oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof.

16. The catalyst of claim 14, wherein the support modifier is selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃.

17. The process of claim 1, wherein the hydrogenation is performed in a vapor phase at a pressure from 10 kPa to 3000 kPa, and a hydrogen to acetic acid mole ratio of greater than 4:1.

18. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

19. A process for producing acetic acid, comprising:
(a) reacting acetic acid and hydrogen in a reactor and in the presence of a catalyst under conditions effective to form a crude ethanol product comprising ethanol, acetic acid, ethyl acetate, and water; and
(b) recovering ethanol from the crude ethanol product;
wherein the reactor is operated at a temperature above a maximum acetic acid evolution temperature, as determined by temperature programmed desorption; and
wherein the reactants have a residence time in the reactor and the reactants are at or above the maximum acetic acid evolution temperature for a majority of the residence time.

20. The process of claim 19, wherein the reactants are at or above the maximum acetic acid evolution temperature for from 5 seconds to 60 seconds.

21. The process of claim 19, wherein the reactants are at or above the maximum acetic acid evolution temperature for from 5 seconds to 25 seconds.

22. The process of claim 19, wherein the maximum acetic acid evolution temperature ranges from 200° C. to 350° C.

23. The process of claim 19, wherein the maximum acetic acid evolution temperature is greater than 280° C.
